(19) United States
(12) Patent Application Publication
(10) Pub. No.: US 2016/0101240 A1
(43) Pub. Date: Apr. 14, 2016

(54) NEEDLE FOR DELIVERING TREATMENT FLUID TO AN AVIAN BIRD, AND ASSOCIATED ASSEMBLY AND METHOD

(71) Applicant: Zoetis Services LLC, Florham Park, NJ (US)

(72) Inventor: William Douglas Samson, Durham, NC (US)

(21) Appl. No.: 14/872,295

(22) Filed: Oct. 1, 2015

Related U.S. Application Data
(60) Provisional application No. 62/061,835, filed on Oct. 9, 2014.

(51) Int. Cl. A61M 5/32; A01K 45/00

(52) U.S. Cl. CPC A61M 5/329; A01K 45/00; A61M 2202/30; A61M 2250/00

(57) ABSTRACT

A needle for delivering a treatment fluid to a target site of an avian bird is provided. Such a needle includes a shaft defining a longitudinal axis and having a proximal end and a distal end. Discrete grooves are defined by the shaft and extend partially along a length thereof. A needle tip extends from the shaft at the distal end thereof. A reservoir assembly includes a body defining first and second holes for receiving the needle therethrough such that the body supports the needle along the length thereof. The reservoir assembly defines a reservoir adapted to receive a treatment fluid through which the grooves pass to carry the treatment substance to the target site. An associated assembly and method are also provided.

### CROSS REFERENCE TO RELATED APPLICATION

This application claims the benefit of U.S. Provisional Patent Application No. 62/061,835, filed Oct. 9, 2014, which is expressly incorporated herein by reference in its entirety.

### TECHNICAL FIELD

The present disclosure generally relates to fluid delivery needles and devices. More particularly, the present disclosure relates to a fluid delivery needle having a plurality of grooves for carrying a treatment fluid to a target site of an avian bird, and an associated assembly and method.

### BACKGROUND

Typically, poultry birds that are raised for protein, egg-laying or breeding purposes may be vaccinated post-hatch against a variety of diseases and parasites. Such vaccinations may prevent debilitation or mortality, while optimizing bird growth and productivity. In many instances, the vaccines or other medicines may be administered manually. In one particular vaccination procedure, a vaccine substance is delivered intradermally in the wing web of an avian bird using, for example, devices disclosed in U.S. Pat. Nos. 2,512,882 and 4,990,135, both to Truesdale, Jr., and U.S. Pat. No. 2,617,418 to Del Pico. Unfortunately, previous and current delivery methods and devices do not provide consistent delivery of the vaccine substance to the wing web in an efficient and reliable manner.

Accordingly, it would be desirable to a needle and delivery assembly capable of providing a consistent volumetric delivery of vaccine substance to a target site of an avian bird. Furthermore, it would be desirable to provide an associated method that would facilitate delivery of a vaccine substance to a target site of an avian bird in a volumetrically consistent and efficient manner.

### BRIEF SUMMARY

The above and other needs are met by aspects of the present disclosure which, according to one aspect, provides a needle for delivering a treatment fluid to an avian bird. The needle includes a shaft defining a longitudinal axis and having a proximal end and a distal end. A plurality of discrete grooves is defined by the shaft and extends partially along a length thereof. A needle tip extends from the shaft at the distal end thereof.

Another aspect provides a delivery assembly for delivering a treatment fluid to an avian bird. The delivery assembly includes a needle adapted to pierce a target site of an avian bird. The needle has a shaft defining a longitudinal axis and has a proximal end and a distal end. The needle has a plurality of discrete grooves defined by the shaft and extending partially along a length thereof. The needle has a needle tip extending from the shaft at the distal end thereof. A reservoir assembly includes a body defining first and second holes for receiving the needle therethrough such that the body supports the needle along the length thereof. The reservoir assembly further defines a reservoir adapted to receive a treatment fluid. An actuator assembly is configured to transport the needle between a home position and an actuated position such that the discrete grooves carry the treatment fluid from the reservoir to the target site of the avian bird.

Yet another aspect provides a method for delivering a treatment substance to a wing web target site of an avian bird. The method comprises providing a needle having a shaft defining a longitudinal axis and having a proximal end and a distal end. The needle includes a plurality of discrete grooves defined by the shaft and extending partially along a length thereof. The needle has a needle tip extending from the shaft at the distal end thereof. The method further comprises actuating the needle such that the discrete grooves pass through a reservoir containing a treatment substance and carry the treatment substance to a wing web target site of an avian bird. The needle tip pierces the wing web target site such that the treatment substance is effectively delivered.

Thus, various aspects of the present disclosure provide advantages, as otherwise detailed herein.

### BRIEF DESCRIPTION OF THE DRAWINGS

Having thus described various embodiments of the present disclosure in general terms, reference will now be made to the accompanying drawings, which are not necessarily drawn to scale, and wherein:

FIG. 1 is a perspective view of a needle for carrying a treatment fluid to a target site, according to one aspect of the present disclosure;

FIG. 2 is a magnified sectional view of the needle of FIG. 1;

FIG. 3 is a side view of the needle illustrated in FIG. 1;

FIG. 4 is a cross-sectional view taken along line 4-4 of FIG. 3;

FIG. 5 is an end view of the needle illustrated in FIG. 1;

FIG. 6 is a cross-sectional view taken along line 6-6 of FIG. 5;

FIGS. 7-9 are perspective views of a reservoir assembly of a delivery assembly, according to one aspect of the present disclosure; and

FIG. 10 is a cross-sectional view taken along line 10-10 of FIG. 9.

### DETAILED DESCRIPTION OF THE DISCLOSURE

Various aspects of the present disclosure now will be described more fully hereinafter with reference to the accompanying drawings, in which some, but not all aspects of the disclosure are shown. Indeed, this disclosure may be embodied in many different forms and should not be construed as limited to the aspects set forth herein; rather, these aspects are provided so that this disclosure will satisfy applicable legal requirements. Like numbers refer to like elements throughout.

The present disclosure provides a needle capable of providing consistent delivery of a fluid substance to a target site of injection. In general, a delivery assembly may be provided to have a fluid holding reservoir 220, a dispensing hole 230 therein, and a needle 100 extending through the reservoir and capable of being reciprocated from a home position with its pointed end 115 substantially contained in the hole 230 to a vaccinating position with its pointed end 115 of a needle tip 120 projecting a distance beyond the hole 230 into the wing web of an avian bird to provide an intradermal injection thereto. The needle 100 may include a proximal end 102 and a distal end 104. The needle tip 120 may be provided at the distal end 104, extending from the shaft 110.

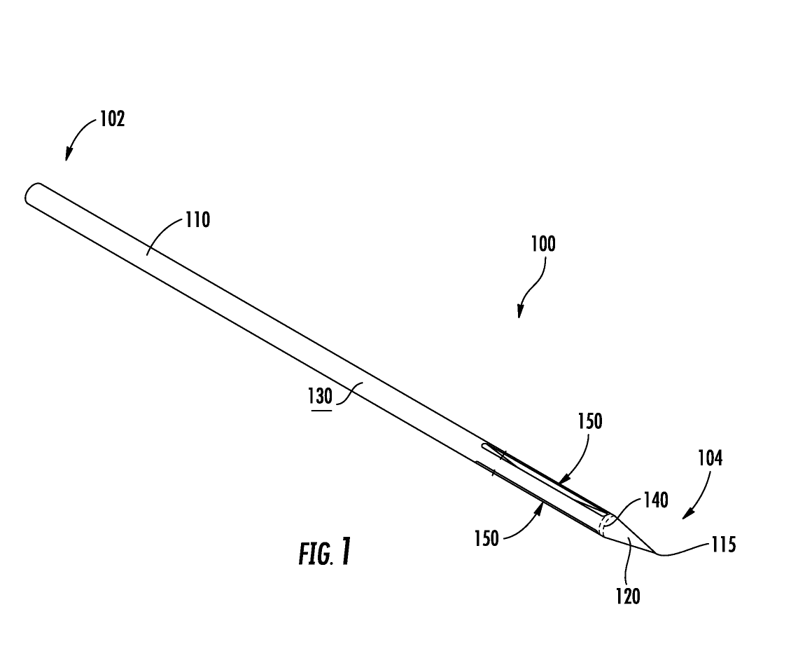

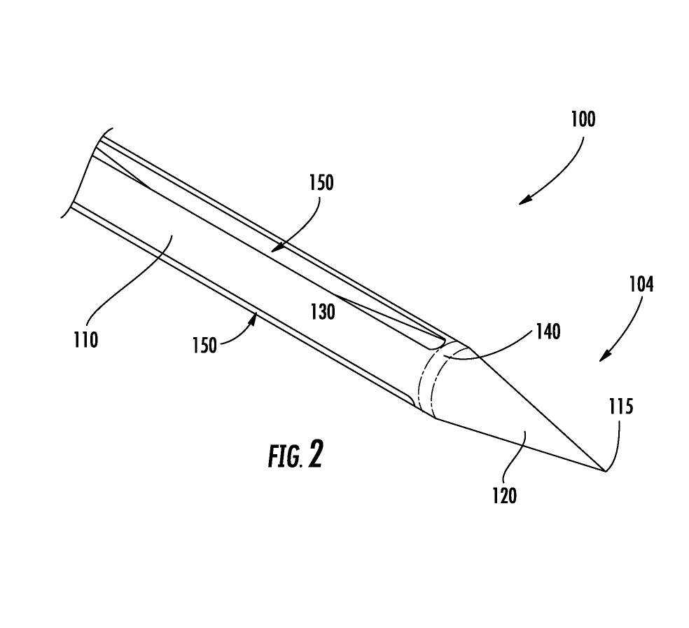

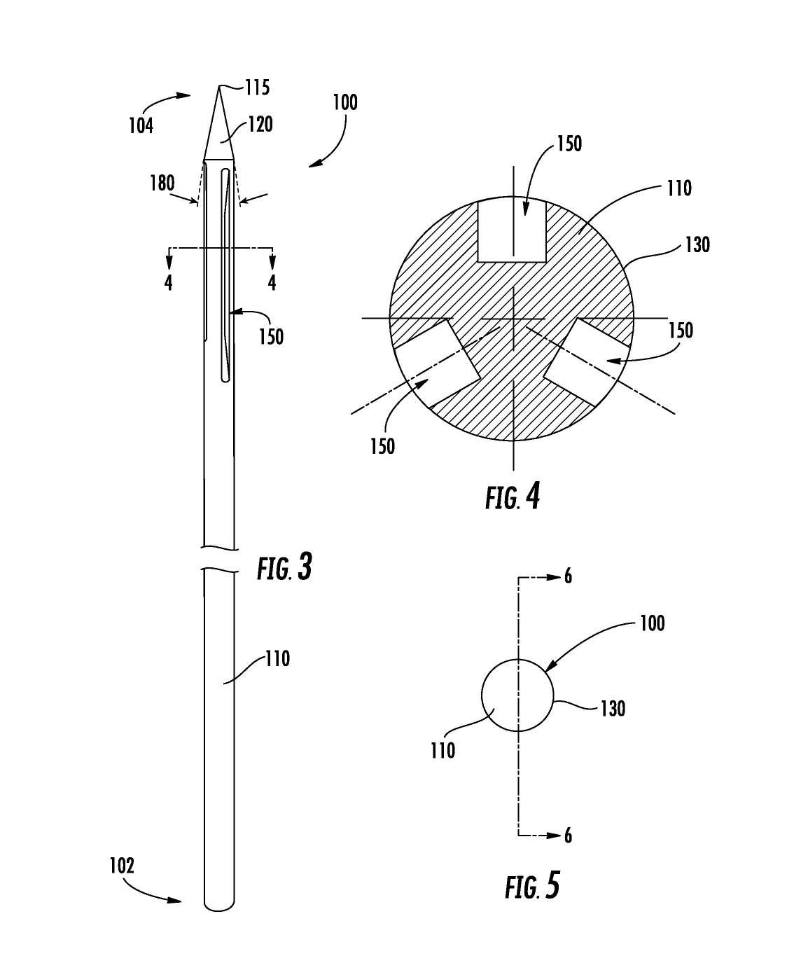

As shown in FIGS. 1-4 and 6, the needle 100 may include a plurality of indentions or grooves 150 defined by a shaft 110 thereof. In some instances, the grooves 150 are discrete and separate from each other such that there is no connection or fluid path linking the individual grooves 150. In this manner, the discrete grooves 150 may act as individual carrier means for carrying the vaccine substance out of the reservoir 220. The grooves 150 may be defined as confined concave indentions of the shaft 110 and configured to contain a volume of liquid. In some instances, the grooves 150 may have a continuous hull shape beginning at an outer surface 130 of the shaft 110 and continuing along a length of the shaft 110 until returning to the outer surface 130. To that end, the grooves 150 may be configured as enclosed and confined liquid holding spaces along the shaft 110. The shaft 110 may be solid with the grooves 150 machined therein by process of removing material from the shaft 110, rather than stamping or deforming the shaft 110 to create indentions.

The grooves 150 may be configured to attract therein a predetermined measured vaccine dose when the grooves 150 are in contact with the reservoir 220 and for releasing the vaccine substance into an avian bird when the pointed end 115 of the needle 100 is inserted therein. The grooves 150 may comprise elongated recesses or indentions that extend partially along the shaft 110 in axial alignment with a longitudinal (central) axis 125 (FIG. 6) of the needle 100. The groove geometry results in a needle 100 that positively attracts the vaccine substance from the reservoir 220 such that the vaccine delivery to the bird is done with reliability.

In this regard, the vaccine substance may be transferred or directed out of the grooves 150 and toward the needle tip 120 when the needle 100 moves from the home position to the vaccination position. That is, the force movement of the needle 100 causes at least some of the vaccine substance carried by the grooves 150 to move out of the grooves 150 toward the pointed end. To achieve such transfer out of the grooves 150, the needle 100 may be advanced to the vaccination position at a velocity of about 20 cm/sec to about 50 cm/sec. Accordingly, the geometrical aspects of the needle 100 of the present disclosure facilitate the vaccine substance being present at the needle tip 120 and pointed end 115 when the pointed end 115 initially pierces (or immediately thereafter) the target site of injection, such as the wing web of an avian bird, thereby achieving a reliable and consistent vaccination of the bird.

According to some aspects, the discrete grooves 150 may be equidistantly spaced about the shaft. In some instances, three discrete grooves 150 may be provided on the shaft 110 and equidistantly spaced apart 120° about the shaft 110. The discrete grooves 150 may be radially disposed about the shaft 110, as shown in FIG. 4. The shaft 110 may define each discrete groove 150 to have first and second end sections 152, 154 sloping from the outer surface 130 of the shaft 110 and transitioning to a substantially flat or planar bottom section 156. In some instances, the first and second end sections 152, 154 may have a curvature or curved profile extending from an end of the bottom section 156 to the outer surface 130 of the shaft 110. The needle 100 may include a neck region 140 shaped to sealingly correspond with the hole 230 so as to seal the vaccine substance within the reservoir 220, while wiping off any excess vaccine substance carried by the needle 100. In some instance, the shaft 110 may be cylindrically shaped to act a sealing means for a reservoir assembly 200, as further explained below. A cross-section of each discrete groove 150 taken perpendicular to the longitudinal axis 125 may have a groove profile having a substantially rectangular shape, as shown in FIG. 4.

The grooves 150 may be geometrically configured to have a volumetric cavity capable of collectively carrying between about 9 microliters to about 12 microliters of a vaccine substance to the target site of injection. Each groove 150 may be geometrically configured to have a volumetric cavity capable of carrying between about 3 microliters to about 4 microliters of a vaccine substance to the target site of injection. According to some aspects, each groove 150 may have a groove length 160 of between about 1 cm and about 2 cm, and particularly between about 1.2 cm and about 1.5 cm. Each groove 150 may have a groove depth of between about 0.4 mm and about 0.6 mm. A length 165 between the neck region 140 and the pointed end 115 may be between about 4 mm and about 8 mm. A length 170 of the bottom section 156 may be between about 4 mm and about 8 mm. A diameter of the shaft 110 may be between about 1.5 mm and about 3 mm. A length of the needle 100 may be between about 7 cm and about 10 cm. An angel 180 of the needle tip 120 with respect to the longitudinal axis 125 may be about 15° to about 20°.

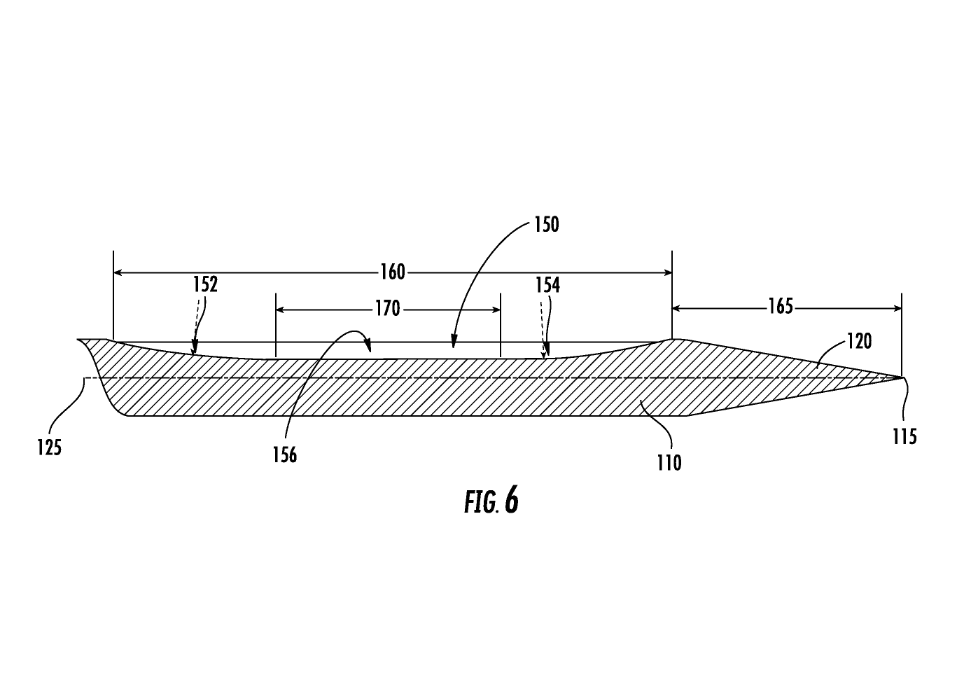

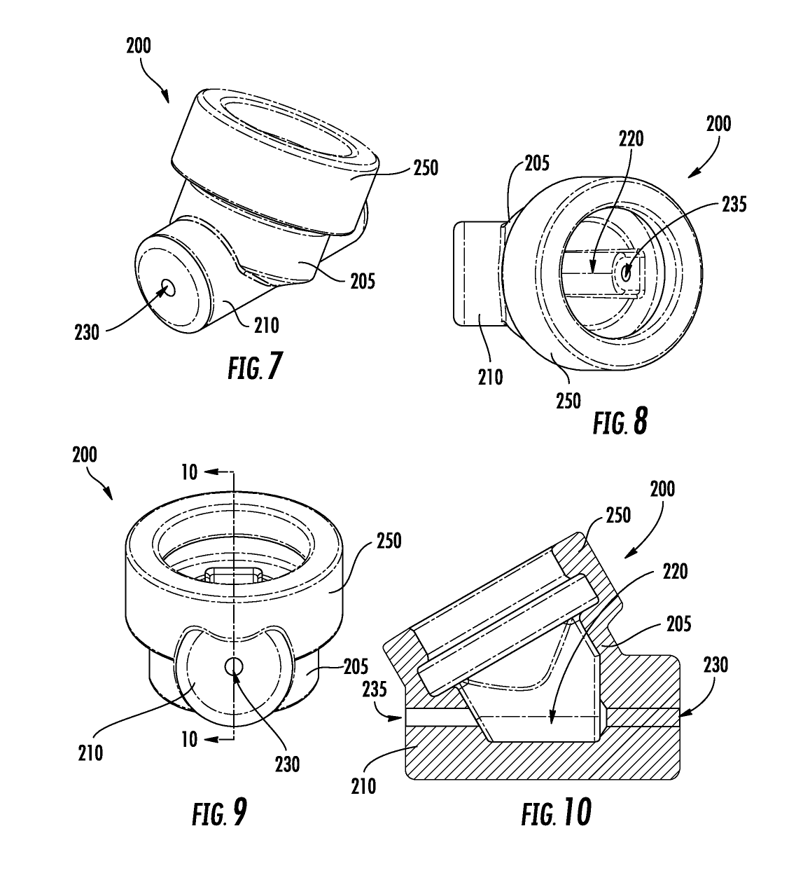

As shown in FIGS. 7-10, a reservoir assembly 200 may hold a vaccine vial (not shown) of a vaccine substance so as to facilitate a quick change out process for spent vials. The reservoir assembly 200 may provide guidance of the needle 100, load the vaccine substance onto the needle 100, and receive and hold the vaccine vial to avoid having to pour vaccine substance from its original container (i.e., the vaccine vial). In this regard, the reservoir assembly 200 may be configured to function as a cap on the vial so as to eliminate the need of transferring the vaccine substance from the vial, thus lessening the risk of cross-contamination and lessening vaccine loss. To that end the reservoir assembly 200 may include a body 205 having a reservoir portion 210 that defines a reservoir 220 and also defines a pair of holes 230, 235 at each end thereof for guiding the needle 100 therethrough to become wetted by passing through the vaccine fluid contained within the reservoir 220. In this regard, loading of vaccine substance into the grooves 150 defined by the shaft 110 of the needle 100 may be accomplished by the needle 100 passing through the reservoir 220 filled with vaccine substance from the vaccine vial naturally by gravity flow (i.e., the vial is upside down such that the vaccine substance flows therefrom naturally into the reservoir 220).

The reservoir portion 210 may also serve as a sealing means around the needle 100 to prevent dripping of vaccine substance from around the needle 100. The reservoir assembly 200 may further include a coupling portion 250 for facilitating attachment of the vaccine vial to the reservoir assembly 200. Thus, receipt and retention of the vaccine vial may be accomplished by the coupling portion 250, which may be in some instances molded to fit directly over a neck of a standard vaccine vial containing vaccine substance used for wing web injections. The vaccine vial may be uncapped and then span fit onto the coupling portion 250.

An actuator assembly (e.g., pneumatic cylinder device) may be provided for moving the needle 100 in a reciprocating manner and within the reservoir assembly 200 between the home position and the vaccination position. In the vaccination position, the wetted needle 100 may be extended to pierce the wing web skin of the avian bird so as to drag the vaccine substance into the tissue thereof.

In use, a vaccine vial may be coupled to the reservoir assembly 200 via the coupling portion 250 in an upside down manner such that the vaccine substance flows into the reservoir 220 via gravity. When it is desired to vaccinate, actuator assembly causes the needle 100 to move from the home position to the vaccination position. In moving to the vaccination position, the grooves 150 move through the reservoir 220 and attract vaccine substance contained in the reservoir 220. The vaccine substance may be carried by the grooves 150 to the target site of injection where the velocity of the needle 100 causes the vaccine substance to be directed toward the needle tip 120 such that the vaccine substance is present at the needle tip during the initial piercing of the wing web skin of the avian bird, thereby reliably and effectively vaccinating the avian bird.

### Example 1

Fifty Cobb 700 broiler breeder pullets were vaccinated in week twelve for fowlpox using Zoetis® Poxine product in Diluent 29. Left and right wing webs received the same vaccine treatment. Vaccination was conducted using a needle and reservoir assembly as disclosed herein. Successful wing web takes (a hard swollen tissue reaction with a wound scab) were evaluated on the tenth day post-vaccination, in week thirteen. Forty-nine of fifty (98%) left wing webs had successful positive takes. The single miss was due to a missed injection. Forty-nine of fifty (98%) right wing webs had successful positive takes. The single miss was attributed to a missed injection at the muscle of the radius bone.

### Example 2

One hundred Cobb 700 broiler breeder pullets were vaccinated in week thirteen for fowlpox using Zoetis® Poxine product in Diluent 29. Left and right wing webs received the same vaccine treatment. Vaccination was conducted using a needle and reservoir assembly as disclosed herein. Successful wing web takes (a hard swollen tissue reaction with a wound scab) were evaluated on the ninth day post-vaccination, in week fourteen. One hundred of one hundred (100%) left wing webs had successful positive takes. Ninety-nine of one hundred (99%) right wing webs had successful positive takes. The single miss was attributed to a missed injection at the muscle of the radius bone.

Many modifications and other aspects of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific aspects disclosed and that modifications and other aspects are intended to be included within the scope of the appended claims. Although specific terms are employed herein, they are used in a generic and descriptive sense only and not for purposes of limitation.

That which is claimed:

1. A needle for delivering a treatment fluid to an avian bird, the needle comprising:
a shaft defining a longitudinal axis, the shaft having a proximal end and a distal end;
a plurality of discrete grooves defined by the shaft and extending partially along a length thereof; and
a needle tip extending from the shaft at the distal end thereof.

2. A needle according to claim 1, wherein the discrete grooves are equidistantly spaced about the shaft.

3. A needle according to claim 2, wherein three discrete grooves are provided on the shaft and equidistantly spaced apart 120° about the shaft.

4. A needle according to claim 1, wherein the discrete grooves are radially disposed about the shaft.

5. A needle according to claim 1, wherein the shaft defines each discrete groove to have first and second end sections sloping from an outer surface of the shaft and transitioning to a substantially flat bottom section.

6. A needle according to claim 1, wherein a cross-section of each discrete groove taken perpendicular to the longitudinal axis provides a groove profile having a substantially rectangular shape.

7. A needle according to claim 1, wherein the discrete grooves are configured to collectively carry about 10 microliters of a treatment substance to a target site.

8. A delivery assembly for delivering a treatment fluid to an avian bird, the delivery assembly comprising:
a needle adapted to pierce a target site of an avian bird, the needle having a shaft defining a longitudinal axis and having a proximal end and a distal end, the needle having a plurality of discrete grooves defined by the shaft and extending partially along a length thereof, and the needle having a needle tip extending from the shaft at the distal end thereof;
a reservoir assembly having a body defining first and second holes for receiving the needle therethrough such that the body supports the needle along the length thereof, the reservoir assembly further defining a reservoir adapted to receive a treatment fluid; and
an actuator assembly configured to transport the needle between a home position and a vaccination position such that the discrete grooves carry the treatment fluid from the reservoir to the target site of the avian bird.

9. A delivery assembly according to claim 8, wherein the reservoir assembly further comprises a coupling arrangement configured to securedly couple a vial containing the treatment fluid to the reservoir assembly.

10. A needle according to claim 8, wherein the discrete grooves are equidistantly spaced about the shaft.

11. A needle according to claim 10, wherein three discrete grooves are provided on the shaft and equidistantly spaced apart 120° about the shaft.

12. A needle according to claim 8, wherein the shaft defines each discrete groove to have first and second end sections sloping from an outer surface of the shaft and transitioning to a substantially flat bottom section.

13. A needle according to claim 8, wherein the discrete grooves are configured to collectively carry about 10 microliters of a treatment substance to a target site.

14. A method for delivering a treatment substance to a wing web target site of an avian bird, the method comprising:
providing a needle having a shaft defining a longitudinal axis and having a proximal end and a distal end, the needle having a plurality of discrete grooves defined by the shaft and extending partially along a length thereof, and the needle having a needle tip extending from the shaft at the distal end thereof; and
actuating the needle such that the discrete grooves pass through a reservoir containing a treatment substance and carry the treatment substance to a wing web target site of an avian bird, the needle tip piercing the wing web target site such that the treatment substance is effectively delivered.
